(19) United States
(12) Patent Application Publication
(10) Pub. No.: US 2007/0073132 A1
(43) Pub. Date: Mar. 29, 2007

(54) APPARATUS AND METHOD FOR MONITORING PATIENTS

(76) Inventor: MICHAEL VOSCH, Titusville, FL (US)

Correspondence Address: ENRIQUE G. ESTEVEZ, 255 SOUTH ORANGE AVE, SUITE 1401, ORLANDO, FL, 32802-3791, US

(21) Appl. No.: 11/535,774

(22) Filed: Sep. 27, 2006

Related U.S. Application Data
(60) Provisional application No. 60/720,997, filed on Sep. 27, 2005.

(51) Int. Cl. A61B 5/04; A61B 5/08; A61B 5/00; A61B 5/103

(52) U.S. Cl. 600/393; 128/903; 600/549; 600/509; 600/595; 600/529

(57) ABSTRACT

An apparatus and method for wireless monitoring of a patient includes a disposable patch having a relatively thin and flexible sealed housing with a surface capable of being releasably attached to the patient's chest; two electrodes positioned spaced apart in said patch, said two electrodes functioning as an EKG lead I by detecting electrical impulses from the patient's heart; a temperature sensor positioned in said patch; a respiration sensor positioned in said patch; an accelerometer capable of detecting a falling motion by the patient; a connector positioned on said patch electrically connected to provide output signals from said two electrodes, said temperature sensor and said respiration sensor; a transceiver connected to said connector to thereby transmit the output signals provided; and a power source connected to supply power to said apparatus.

### RELATED APPLICATION

This application claims priority from co-pending U.S. provisional application Ser. No. 60/720,997, which was filed on Sep. 27, 2005, and which is incorporated herein by reference in its entirety.

### FIELD OF THE INVENTION

This invention relates to the field of medical monitoring and, more specifically, to a disposable electrode patch and apparatus for wireless monitoring of debilitated individuals and, particularly, the elderly patient.

### BACKGROUND OF THE INVENTION

The elderly population is growing in numbers, particularly in the United States. It is well known that the elderly, as well as other types of debilitated patients, are prone to falling. Additionally, for many elderly who are living in group homes or other care facilities such as assisted living centers, it would be desirable to have a system which monitors the person's physical parameters with a minimum of intrusion and without curtailing the person's mobility due to connecting wires. A monitoring system that would also generate an alarm in response to the person falling, would be desirable for use with the elderly patient.

With the foregoing in mind, the present invention provides a disposable patch which adheres to the patient and picks up predetermined electrical impulses or other bodily signals whereby to monitor the patient's physical status, including, for example, heart function, respiration and temperature. The patch also contains a sensor which will generate an alarm signal if the patient is sensed to fall, thereby summoning aid.

### SUMMARY OF THE INVENTION

With the foregoing in mind, the present invention advantageously provides an apparatus which includes a disposable patch having a plurality of electrodes and sensors adapted for detecting predetermined electrical impulses and/or signals from the patient, these providing output data associated with one or more of the person's physiological parameters, including heart function, respiration rate, and temperature. The apparatus preferably also includes an accelerometer. As known to the skilled, an accelerometer is an electromechanical device that will measure acceleration forces. The force may be static, such as the constant force of gravity, or could be dynamic, such as the force generated by motion or vibration. Measuring acceleration caused by gravity would enable the system to sense how the device is moving and, particularly, at what angle the device is tilted at with respect to the ground. Accordingly, the device would be able to indicate when the patient may be falling and responsively generate an alarm signal to warn care personnel that the patient may need aid. One preferred embodiment of the invention includes a power source, such as a nine (9) Volt battery associated with a reusable wireless transceiver/antenna which is connected to the disposable patch containing the electrodes and sensors.

The device wirelessly communicates with a base station which could be a personal computer (PC), a personal digital assistant device (a PDA), orsimilar machine. The patch/transceiver would transmit data containing basic patient information, for example heart function, by a standard wireless protocol such as Bluetooth or 802.11. Communication between the patch/transceiver apparatus worn by the patient and a central base station could be programmed to occur at predetermined periodic intervals, could be responsive to polling by the central base station, or could be continuously transmitted in a data stream. The alarm signal described above may be audible, visual or both and may be generated from the transceiver unit, from a base station, or from both. The alarm triggering signal, along with an identifier for the specific patch/transceiver, may even be broadcast to one or more portable wireless devices such as a pager or a PDA carried by attending staff in the facility.

The present system would be able to monitor a large number of patients at a time. Accordingly, the system is well suited for monitoring patients in a nursing home for basic physiological parameters and for falls. Each electrode or sets of electrodes on the disposable patch would collect input electrical signals from the patient and then, either routinely or when queried from the base station (PC/PDA), would download to the base station display preferably in web page format the patient's physiological parameters and, optionally, the patch wold also indicate battery status. The system would preferably employ a standard TCP/IP protocol and wireless 802.11g but other protocols could be used as well. As illustrated in FIG. 1, preferred patient parameters to be monitored include pulse or heart rate, temperature by way of a thermistor or other similar device, respiration rate by way of a strain gauge or other similar device, and falls via the accelerometer. In the patch as shown in FIG. 2, E3 and E4 are used to determine the patient heart function and are the equivalent of an EKG lead I, thereby being capable of generating what is commonly called a “rhythm strip” type of graphic record showing heart function. E1 in the diagram is a temperature sensor, such as a thermistor, and E2 is a respiration rate sensor, such as a strain gauge. The connector is used to connect the disposable patch portion with the reusable transceiver and battery portion of the system.

### BRIEF DESCRIPTION OF THE DRAWINGS

Some of the features, advantages, and benefits of the present invention having been stated, others will become apparent as the description proceeds when taken in conjunction with the accompanying drawings in which:

FIG. 1 is a schematic diagram of an embodiment of the disposable patch of the present invention, connectable to a microprocessor, transceiver and antenna, according to an embodiment of the present invention;

FIG. 2 depicts a complete disposable patch of the invention, including all components of the presently described apparatus; and

FIG. 3 is a flow diagram illustrating a method of the invention.

### DETAILED DESCRIPTION OF THE PREFERRED EMBODIMENT

The present invention will now be described more fully hereinafter with reference to the accompanying drawings, in which preferred embodiments of the invention are shown. This invention may, however, be embodied in many different forms and should not be construed as limited to the illustrated embodiments set forth herein. Rather, these illustrated embodiments are provided so that this disclosure will be thorough and complete, and will fully convey the scope of the invention to those skilled in the art.

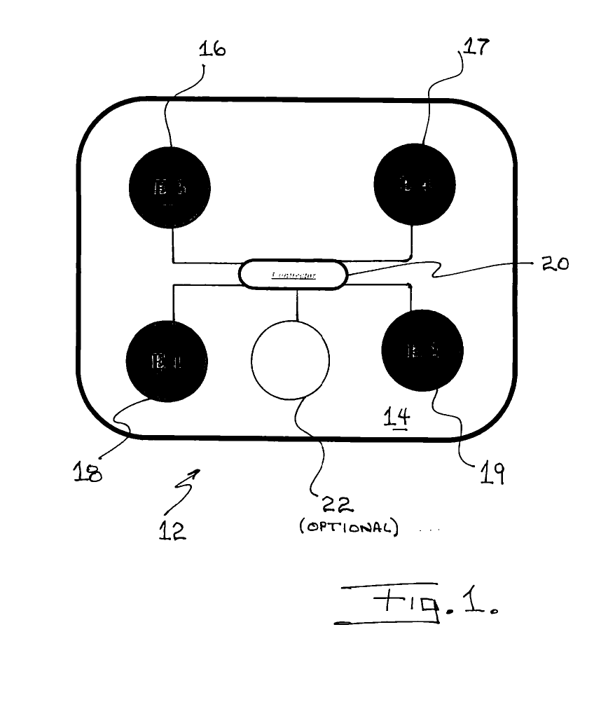

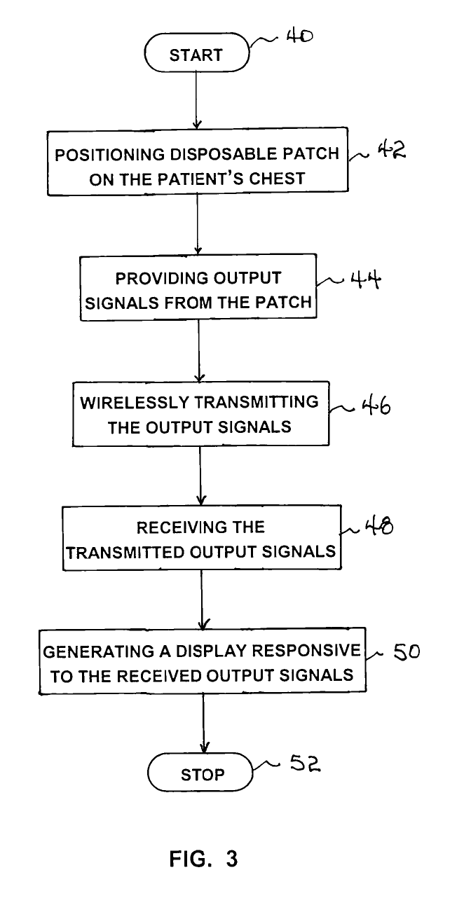

FIGS. 1-3 illustrate an apparatus 10 and method for monitoring a patient for a plurality of physical parameters. The present apparatus 10 includes a disposable patch 12, as shown in FIG. 1, having a relatively thin and flexible sealed housing 14 with a surface capable of being releasably attached to the patient's chest. Two electrodes 16, 17 are positioned spaced apart in said housing, said two electrodes functioning as an EKG lead I by detecting electrical impulses from the patient's heart. A temperature sensor 18 and a respiration sensor 19 are positioned in said housing. A connector 20 associated with an outer surface of said patch is electrically connected to provide output signals from said two electrodes 16,17, said temperature sensor 18 and said respiration sensor 19.

One preferred embodiment of the apparatus includes an adhesive disposed along at least a portion of an outer surface of the housing 14, said adhesive effective for releasably attaching said disposable patch 12 to the patient's chest. More preferably, a water resistant adhesive is disposed along at least a portion of an outer surface of the housing 14, said adhesive effective for releasably attaching said disposable patch to the patient's chest in a substantially water resistant manner.

Additional sensors in the apparatus include a temperature sensor 18 which comprises a thermistor. The respiration sensor 19 preferably comprises a strain gauge and may comprise a piezoresistive material, which the skilled will recognize as capable of generating an electrical current responsive to mechanical strain applied to the material. The connector 20 is substantially resistant to moisture, so that a substantially water resistant connection can be maintained.

A preferred embodiment of the present apparatus, as shown in FIG. 1, also includes an optional accelerometer 22 associated with said disposable patch 12. Those skilled in the art will recognize that an accelerometer 22 is a known device for detecting motion and, particularly, rapid motion such as occurs during a fall by the patient. There may be more than one type of motion detection device suitable for use in the invention and, therefore, the term “accelerometer” is intended to include those devices as well.

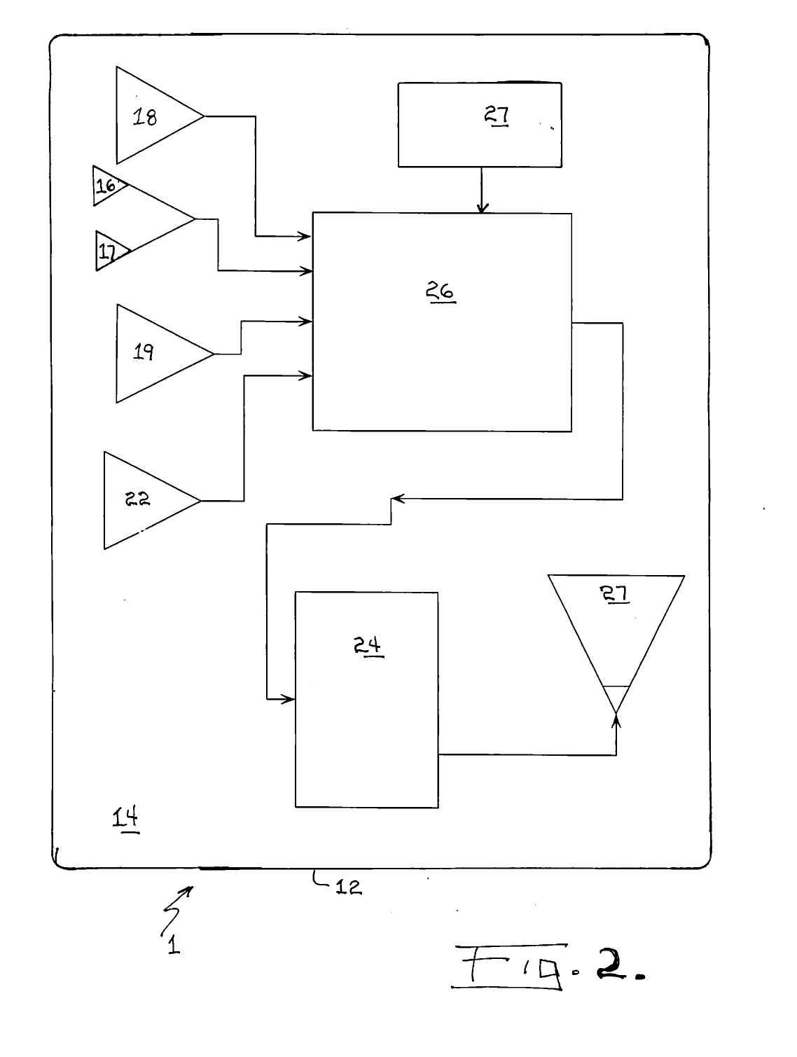

Another embodiment of the present invention, depicted in FIG. 2, includes an apparatus 10 for wireless monitoring of a patient. The apparatus includes a disposable patch 12 having a relatively thin and flexible sealed housing 10 with a surface capable of being releasably attached to the patient's chest, two electrodes 16, 17 positioned spaced apart in said patch, said two electrodes functioning as an EKG lead I by detecting electrical impulses from the patient's heart, a temperature sensor 18 positioned in said patch, a respiration sensor 19 and an accelerometer 22 are positioned in said patch. A connector 20 may be positioned on said patch, as illustrated in FIG. 1, electrically connected to provide output signals from said two electrodes 16, 17, said temperature sensor 18, said respiration sensor 19 and the accelerometer 22. More preferably, in an embodiment of the invention as shown in FIG. 2, the patch contains all the components of the invention, for example, the accelerometer 22, a transceiver 24, a microprocessor 26 to thereby obtain and process output signals; an antenna 27; and a power source 28 connected to supply power to said apparatus. The various features of this embodiment are as set forth above with respect to the disposable patch.

A method aspect of the present invention includes, from the start 40, positioning a disposable patch 42 having a relatively thin and flexible sealed housing on the patient's chest, the patch having two electrodes 16, 17 positioned spaced apart so as to function as an EKG lead I, having a temperature sensor 18, a respiration sensor 19, an accelerometer 22 and a patch connector 20 electrically connected, thus providing 44 output signals from the two electrodes, the temperature sensor, the respiration sensor and the accelerometer. The method continues by wirelessly transmitting 46 the output signals, followed by receiving 48 the transmitted output signals. Thereafter the method continues by generating a display 50 responsive to the received output signals. Thereafter, the method stops 52.

In the method, wirelessly transmitting 46 may include a standard wireless protocol and may preferably include a protocol selected from Bluetooth and 802.11. Receiving 48 the transmitted output signals preferably comprises a device selected from a personal computer, a personal digital assistant, a pager, a cellular telephone, and combinations thereof. Also, wirelessly transmitting 46 may be selected to occur at predetermined time intervals, responsive to polling by a central base station, continuously streaming, and in combinations thereof. The method may further comprise optionally generating an alarm responsive to a predetermined output signal. The skilled will recognize that the method may be employed so that multiple patients are monitored substantially simultaneously and where receiving may occur at a base station remote from the patient's location. Generating a display may be effected through a computer network or through a global computer network such as the internet.

Accordingly, in the drawings and specification, there have been disclosed a typical preferred embodiment of the invention, and although specific terms are employed, the terms are used in a descriptive sense only and not for purposes of limitation. The invention has been described in considerable detail with specific reference to these illustrated embodiments. It will be apparent, however, that various modifications and changes can be made within the spirit and scope of the invention as described in the foregoing specification and as defined in the appended claims.

That which is claimed:

1. An apparatus for monitoring a patient for a plurality of physical parameters, said patch comprising:
a disposable patch having a relatively thin and flexible sealed housing with a surface capable of being releasably attached to the patient's chest;
two electrodes positioned spaced apart in said housing, said two electrodes functioning as an EKG lead I by detecting electrical impulses from the patient's heart;
a temperature sensor positioned in said housing;
a respiration sensor positioned in said hoursing; and
a connector associated with an outer surface of said patch and being electrically connected to provide output signals from said two electrodes, said temperature sensor and said respiration sensor.

2. The apparatus of claim 1, further comprising an adhesive disposed along at least a portion of an outer surface of the housing, said adhesive effective for releasably attaching said disposable patch to the patient's chest.

3. The apparatus of claim 1, further comprising a water resistant adhesive disposed along at least a portion of an outer surface of the housing, said adhesive effective for releasably attaching said disposable patch to the patient's chest in a substantially water resistant manner.

4. The apparatus of claim 1, wherein said temperature sensor comprises a thermistor.

5. The apparatus of claim 1, wherein said respiration sensor comprises a strain gauge.

6. The apparatus of claim 1, wherein said respiration sensor comprises a piezoresistive material.

7. The apparatus of claim 1, wherein said connector is substantially resistant to moisture.

8. The apparatus of claim 1, further comprising an accelerometer associated with said disposable patch.

9. An apparatus for wireless monitoring of a patient, said apparatus comprising:
a disposable patch having a relatively thin and flexible sealed housing with a surface capable of being releasably attached to the patient's chest;
two electrodes positioned spaced apart in said patch, said two electrodes functioning as an EKG lead I by detecting electrical impulses from the patient's heart;
a temperature sensor positioned in said patch;
a respiration sensor positioned in said patch;
an accelerometer capable of detecting a falling motion by the patient;
a connector positioned on said patch electrically connected to provide output signals from said two electrodes, said temperature sensor and said respiration sensor;
a transceiver connected to said connector to thereby wierelessly transmit the output signals provided; and
a power source connected to supply power to said apparatus.

10. The apparatus of claim 9, further comprising an adhesive disposed along at least a portion of an outer surface of said disposable patch, said adhesive effective for releasably attaching said patch to the patient's chest.

11. The apparatus of claim 9, further comprising a water resistant adhesive disposed along at least a portion of an outer surface of the housing, said adhesive effective for releasably attaching said disposable patch to the patient's chest in a substantially water resistant manner.

12. The disposable patch of claim 9, wherein said temperature sensor comprises a thermistor.

13. The disposable patch of claim 9, wherein said respiration sensor comprises a strain gauge.

14. The disposable patch of claim 9, wherein said respiration sensor comprises a piezoresistive material.

15. The disposable patch of claim 9, wherein said connector is substantially resistant to moisture.

16. A method of monitoring a patient, the method comprising:
positioning a disposable patch having a relatively thin and flexible sealed housing on the patient's chest, the patch having two electrodes positioned spaced apart to function as an EKG lead I, having a temperature sensor, a respiration sensor, an accelerometer and a patch connector electrically connected to provide output signals from the two electrodes, the temperature sensor, the respiration sensor and the accelerometer;
wirelessly transmitting the output signals;
receiving the transmitted output signals; and
generating a display responsive to the received output signals.

17. The method of claim 16, wherein wirelessly transmitting includes a standard wireless protocol.

18. The method of claim 16, wherein wirelessly transmitting includes a standard wireless protocol selected from Bluetooth and 802.11.

19. The method of claim 16, wherein receiving the transmitted output signals comprises a device selected from a personal computer, a personal digital assistant, a pager, a cellular telephone, and combinations thereof.

20. The method of claim 16, wherein transmitting is selected to occur at predetermined time intervals, responsive to polling by a central base station, continuously streaming, and in combinations thereof.

21. The method of claim 16, further comprising generating an alarm responsive to a predetermined output signal.

22. The method of claim 16, wherein multiple patients are monitored substantially simultaneously.

23. The method of claim 16, wherein receiving occurs at a base station remote from the patient's location.

24. The method of claim 16, wherein generating a display is effected through a computer network.

25. The method of claim 16, wherein generating a display is effected through a global computer network.
